(19) United States
(12) Patent Application Publication
(10) Pub. No.: US 2025/0069744 A1
(43) Pub. Date: Feb. 27, 2025

(54) SYSTEM AND METHOD FOR MEDICAL DISEASE DIAGNOSIS BY ENABLING ARTIFICIAL INTELLIGENCE

(71) Applicant: Nikhil Madan, Fremont, CA (US)

(72) Inventor: Nikhil Madan, Fremont, CA (US)

(21) Appl. No.: 18/238,453

(22) Filed: Aug. 25, 2023

(51) Int. Cl. G16H 50/20; G16H 10/60; G16H 15/00; G16H 30/20; G16H 30/40; G16H 50/70

(52) U.S. Cl. CPC G16H 50/20; G16H 10/60; G16H 15/00; G16H 30/20; G16H 30/40; G16H 50/70

(57) ABSTRACT

A medical image disease diagnostic system that is included in a clinical medical diagnosis workflow. In one embodiment, a user interface is configured to assess a medical imaging application that provides for viewing, analyzing, and annotating of medical images and medical non-image data and medical training data. A processor includes a pre-trained subsystem configured to generate AI pre-trained models based on the medical image and medical non-image data, a training data generation subsystem configured to generate the medical training data, and a medical image model training subsystem configured to transform the medical image and medical non-image data into the medical training data. The medical image model training subsystem is further configured to train or generate the pre-trained AI models based on the medical training data, and a model drift subsystem is configured to determine accuracy of the pre-trained AI models. A diagnostic reporting subsystem configured to generate clinical diagnostic reports.

### CROSS-REFERENCE TO RELATED APPLICATIONS

This patent claims the benefit of U.S. Provisional Patent Application 63/532,350, filed Aug. 12, 2023, titled System and Method for Medical Disease Diagnosis by Enabling Artificial Intelligence. The entire content of each afore-listed earlier-filed application is hereby incorporated by reference for all purposes.

### FIELD OF THE INVENTION

The present disclosure generally relates to computer-implemented medical image processing and interpretation techniques using artificial intelligence, segmented images and annotated training data for machine learning and bi-directional interaction between computer interfaces and clinicians for medical diagnosis.

### BACKGROUND

The healthcare industry, particularly radiology and clinical diagnostics, has countless opportunities to leverage Artificial Intelligence (AI) and machine learning to achieve more precise, proactive, and complete patient diagnosis. There are approximately two trillion medical images produced globally, with ˜50% in the United States, every year. This vast number includes a wide range of imaging modalities, such as X-rays, CT scans, MRI scans, and ultrasound, among others. These images have historically doubled every five years, and are rapidly accelerating, however, there is a lack of clinicians, in particular radiologists, to support the growing number of medical images. AI-based solutions, such as deep learning models and computer vision algorithms are increasingly being used in medical imaging to enable faster diagnosis of imaging, make the evaluation mode reproductible and consistent, and increase diagnostic accuracy to help case the burden of clinicians and improve health outcomes for patients. Despite the tremendous benefits of AI technology in medical imaging, it has not yet been widely adopted and the process of building and deploying medical imaging AI is fragmented.

### SUMMARY

The following presents a summary to provide a basic understanding of one or more embodiments of the invention. The following is a non-exhaustive listing of some aspects of the present techniques. These and other aspects are described in the following disclosure. This summary is not intended to identify key or critical elements or delineate any scope of the different embodiments or any scope of the claims. Its sole purpose is to present concepts in a simplified form as a prelude to the more detailed description that is presented later. In one or more embodiments, systems, computer-implemented methods, apparatus and/or computer program products are described herein that facilitate the integration of artificial intelligence into medical imaging systems using a distributed deep learning platform.

Some aspects include a medical image disease diagnosis system, including: a memory that stores computer executable components; and a processor that executes the computer executable components stored in the memory, wherein the computer executable components include: a user interface module that facilitates accessing a medical imaging application that provides for one or more activities of viewing, analyzing, monitoring or sharing a plurality of medical images, wherein the medical images include medical training data; a training data generation subsystem that facilitates generating medical training data associated with viewing the plurality of medical images using the imaging application, wherein the medical training data includes labeling data that defines one or more attributes in the plurality of medical images and wherein the training data generation module includes: a medical image receiving module that provides the medical training data to one or more machine learning systems, wherein the one or more machine learning systems is configured to develop or train one or more medical diagnostic models from the medical training data and provide artificial intelligence-generated medical diagnostic analysis of the plurality of medical images; and a labeling module that generates the labeling data based on a labeling mark applied to the one or more attributes in the plurality of medical images as displayed via a graphical interface.

Building medical models is a lengthy and complex task with significant bottle necks throughout the process. Currently, the process involved in building AI is done outside clinical workflow and often disjoined and siloed. The challenges of medical diagnosis outside of the clinical workflow include collecting data from various sources, such as hospitals or imaging centers. This is often characterized as siloed data storage and limited interoperability between different Picture Archiving and Communication Systems (PACS), privacy and compliance management of patient medical imaging data and medical information data, labeling and annotating medical imaging data through a certified radiologist panel to ensure accuracy, choosing the type of AI model that best suits clinical needs, followed by training the model using pre-processed labeled data. Additionally, testing and evaluating performance (Specificity/sensitivity) of AI models on real world clinical dataset and workflow, integrating proprietary or third-party AI model into existing medical imaging software or creating a standalone application, and monitoring AI models to ensure it continues to perform effectively are ongoing challenges.

There lacks a system-based approach to resolve the forementioned challenges in one integrated platform that provides a full loop process encompassing data collection, preparation/labeling, and AI development, deployment, monitoring, and continuous iteration.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

Embodiments are illustrated by way of example and not limited to the figures of the accompanying drawings in which like references indicate similar elements.

FIG. 1 illustrates a schematic architectural view of a medical image disease diagnostic system in a real-time clinical workflow, according to an embodiment of the present invention;

FIG. 2 illustrates a block diagram of a medical image disease diagnostic system, according to an embodiment of the present invention;

FIG. 3 illustrates a schematic view of the training, augmentation, and adjudicating in the system of FIGS. 1 and 2;

FIG. 4 illustrates a schematic view of evaluating and improving pre-trained models in the system of FIGS. 1 and 2;

FIG. 5 illustrates a schematic view of developing and retraining diagnostic reporting models in the system of FIGS. 1 and 2;

FIG. 6A to 6C present medical imaging visualizations generated associated with *** with a medical imaging application in accordance with an embodiment of the present invention;

FIG. 7 illustrates a general workflow of the system of FIG. 2.

FIG. 8 illustrates a detailed workflow of the subsystem of FIG. 5.

### DETAILED DESCRIPTION

To mitigate problems herein, the inventors had to both invent solutions and, in some cases, just as importantly, recognize problems overlooked (or not yet foreseen) by others in the field of artificial intelligence. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Reference in the specification to “one embodiment” or “an embodiment” means that a particular feature, structure, or characteristic described in conjunction with the embodiment can be included in at least one embodiment. The appearances of the phrase “in one embodiment” in various places in the specification do not necessarily refer to the same embodiment.

In healthcare, machine learning models are used to assist with tasks such as diagnosing diseases, predicting patient outcomes, and identifying patients at risk for certain conditions. However, before a machine learning model can be used for these purposes, it must be trained on a large dataset of patient data. This may be done through a process called annotation, in which human experts review and label medical data to indicate which patients have certain conditions or outcomes. One of the greatest challenges is the annotation process is manual and must be done by trained professionals with expertise in the relevant medical domain which makes it time-consuming and costly. Furthermore, the process of building and refining machine learning models often involves multiple iterations of data collection, annotation, and model development, which further increases time and costs. Additionally, the annotation process is often completed outside of the clinical workflow. Experts who are responsible for reviewing and labeling the data may not have access to all the relevant clinical information, nor be able to integrate their annotations seamlessly into the clinical workflow of patients.

Not only is manual, costly, and outside of clinical workflow an issue, but reduced quality of the labeled medical imaging data for AI training is also a major factor in the performance and reliability of AI algorithms. Some of the common quality issues in labeling medical images for AI training are interobserver variability, ambiguity and uncertainty, lack of consensus or ground truth, and variability in image quality.

The first quality issue, interobserver variability, is caused by different clinicians, i.e., radiologists, may interpret and label images differently due to subjective factors or varying levels of expertise. This leads to inconsistencies in the labeled data and affecting the training and evaluation of AI algorithms.

Another quality issue is the presentation of ambiguous findings or unclear boundaries within medical images. This ambiguity can introduce uncertainty and affect the accuracy of AI training algorithms on medical image data. Additionally, not having definitive ground truth and consensus among experts for certain medical conditions or abnormalities makes it challenging to establish accurate labels for training AI algorithms. Lastly, variability in image quality, i.e., resolution, quality, noise, or artifacts may create difficulties in identifying and labeling certain features accurately, therefore impacting the reliability of the labeled data.

In addition to manual processes, outside of clinical workflow and quality issues, data scarcity of positive cases causes challenges to build machine learning models with high sensitivity and specificity. In the context of medical imaging, the data scarcity of positive cases can be a significant challenge when building AI models for disease diagnosis. For instance, if the prevalence of a certain medical condition is low, the number of positive cases in the dataset may be limited. This instance may lead to a situation where the AI model is trained on an imbalanced dataset, with a much larger number of negative cases than positive cases. In this scenario, the AI model may struggle to identify positive cases accurately, leading to a high false negative rate. This can be particularly problematic when dealing with life-threatening conditions, such as cancer, where early detection and accurate diagnosis are critical.

Furthermore, the scarcity of positive cases can also affect the sensitivity and specificity of the AI model, as it may be more prone to false positive predictions due to a lack of positive samples in the training data. False positive lead to unnecessary follow-up tests and treatments, which are costly and harmful to patients.

As machine learning models are trained, model drift causes inaccuracy and reduces the reliability of diagnosis. Model drift is a situation where the performance of a machine learning model decreases over time due to changes in the distribution of the data. This happens for several reasons, including changes in the patient population, imaging equipment or clinical protocols. A decrease in model performance leads to missed diagnoses or incorrect treatment plans, which both have serious consequences for patients.

Lasting in the field of AI within the medical diagnosis industry, summarizing findings from a medical imaging diagnosis can be ineffective and error prone. In many cases, clinician diagnostic reports, including teleradiology reports, are generated manually or with the help of scribes, which can be time-consuming and prone to errors. Radiologists and other healthcare providers and clinicians need to manually enter data into a reporting system, which leads to transcription errors and/or delays in report generation. Radiology reports vary widely in terms of format, content, and style, which make it difficult for healthcare providers and clinicians to interpret and act upon the information in the report. This lack of standardization makes it difficult to compare and analyze data across different healthcare providers and settings.

One or more embodiments of the disclosed subject matter are directed to systems, computer-implemented methods, apparatus, and/or computer programs that facilitate integrating AI into the clinical workflow of diagnosis medical conditions using a distributed learning platform and architecture. The distributed learning architecture can connect hardware and software needed for developing trained machine learning models and applying the models directly into the clinical workflow. The AI architecture can further facilitate an ongoing continuously maintained, monitored, and improved model optimization and expansion based on model evaluation and correction. The AI architecture can integrate machine learning assisted labeling systems and processes into real-time clinical workflow. The system further assists in the development of models that are more tailored to the needs of clinicians and integrates effectively within existing real-time clinical workflows.

One or more embodiments of the distributed learning architecture incorporates adjudication processes that compare diagnosis of multiple clinicians and/or domain experts involved in adjudication to reach a consensus to resolve complex cases. Additionally, the adjudication process provides guidance on ambiguous findings, and review the labeled data for gradeability, accuracy, and clinical relevance. The adjudication is decided on the nature of the labeling tasks. Various labeling tasks, including, but not limited to, detection, classification, and quantification will require a different approach to adjudication. Judge based adjudication requires an independent expert to weigh in and provide an adjudication of the labels produced by individual clinicians. Majority rule for detection/classification tasks requires a majority vote to be taken in cases n>2 labelers with odd number of clinicians used to break symmetry. Mean/median approaches are used for quantification tasks. For example, calculi (kidney stones) diameter measurement can be used to reduce noise. Additionally quality control measures are put in place to ensure that the data used to train the machine learning models is of high quality and consistent over time. This involves data curation tools and techniques to remove low-quality or inconsistent data with high degree of variability during the adjudication process.

In various embodiments, the distributed learning architecture within real-time clinical workflow uses data augmentation and synthetic data generation techniques to generate more diverse training data, reduce overfitting, and improve the generalization capability of the AI model. To generate multimodal synthetic data used within the distributed learning architecture, various methods are used.

One or more embodiments of the distributed learning architecture within real-time clinical workflow address model drift by regular retraining and versioning of the models, continuous monitoring of the performance of the machine learning model to detect changes in performance and apply domain adaptation techniques to adapt the machine learning model to new imaging equipment or clinical protocols. Regular retraining and versioning of the models is achieved by addressing model drift. This is to retrain the machine learning model regularly using new medical imaging data flowing through the one or more embodiments. This approach helps the model to adapt to changes in the distribution of the data and maintain high performance over time.

Continuous monitoring of the performance of the machine learning model to detect changes in performance early and take corrective action before the model performance drops significantly is essential and incorporated in one or more of the embodiments. Lastly, domain adaptation techniques can be used to adapt the machine learning model to new imaging equipment of clinical protocols. In this instance, this can involve using transfer learning techniques or fine-tuning the model on new data. Additionally, optimization of radiologists and clinician inputs is optimized using prompt engineering to generate natural language summaries or recommendations through Generative AI.

One or more embodiments are now described with reference to the drawings, wherein like referenced numerals are used to refer to like elements throughout. In the following description, for purposes and explanation, numerous specific details are set forth in order to provide a more thorough understanding of one or more embodiments. It is evident, however, in various cases, that one or more embodiments can be practiced without these specific details.

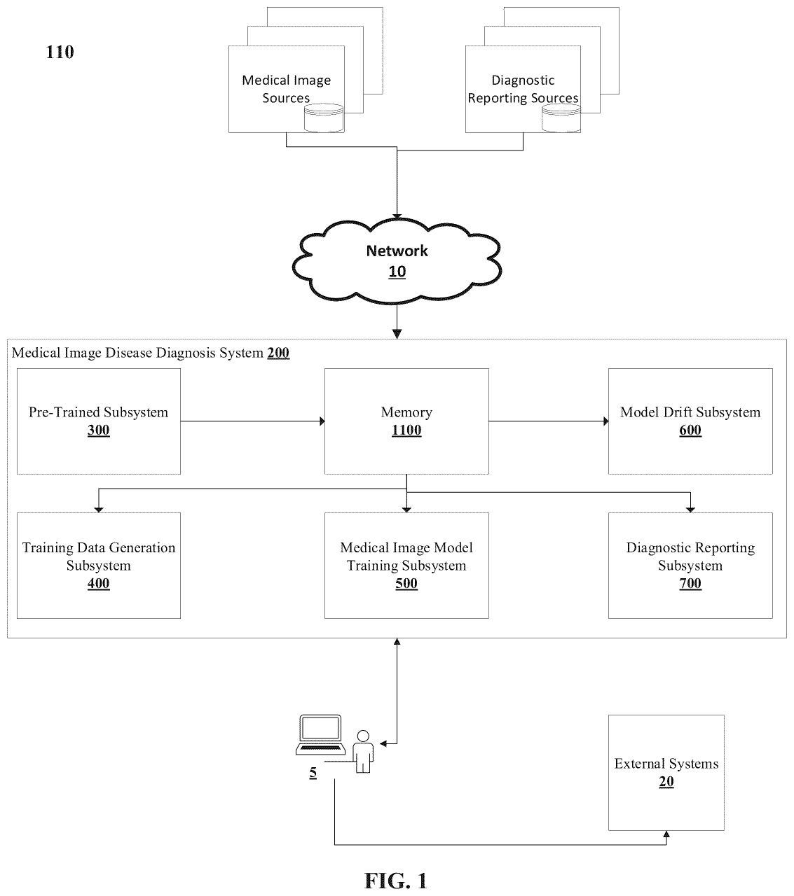

FIG. 1 is a schematic architectural view of a medical image disease diagnostic system in a real-time clinical workflow 110. The system 200 includes a pre-trained subsystem 300, a training data generation subsystem 400, a medical image model training subsystem 500, a model drift subsystem 600, and a diagnostic reporting subsystem 700. The medical image disease diagnosis is connected to a user interface that includes a graphical user interface (GUI), not shown that one or more users 5 interact with. The user interface is connected to external systems 20. The medical image disease diagnosis system 200 receives medical images, medical information, and diagnostic reporting information from various sources through a network 10 and is stored within memory 1100. Network 10 may be a local area network (LAN), a metropolitan area network (MAN), a wide area network (WAN) such as the Internet or intranet, a private cloud network, a public cloud network, or combination thereof.

Medical image and patient information sources can include medial image data in a DICOM (digital imaging and communications in medicine) format, natural medical image data, and annotated medical images, medical image data in a non-DICOM format, scheduling data, demographic data, insurance data, billing data, registration data, prescription data, workflow data, EKG data, and medical history data.

Clinicians rely on data from various sources to incorporate into medical diagnostic reports. The data can be metadata from the various sources that are used to train, retain, and/or fine-tune AI training models for the development of diagnostic reports. refresh machine learning models. These metadata sources can be foundational language, medical domain knowledge, or medical knowledge graphs, or combination thereof.

Foundational language models are generalized in nature; therefore, they are not inherently adapted to specified domains. The foundational language models are trained on generic texts, images, speech, structured data, or 3D signals, and any combination thereof. These models can be fine-tuned by using specific domain data associated with defined industries, such as medical diagnostics.

Domain knowledge is expertise knowledge that is tailored to a specific industry, subject, tasks, solution, etc. In the instance of the present invention, medical domain knowledge is tailored to the medical domain or field, more specifically, medical diagnostic. The medical domain knowledge can include medical journals, articles, whitepapers, presentations, books, textbooks, and expert speeches or discussions. This specific knowledge domain data can be applied to foundational models to adjust the model metadata to be specific to the medical field or industry.

Additionally, medical diagnostic report data can include specified knowledge graphs in the medical industry or domain. In the instance of the present invention, medical knowledge graphs are associated specifically with the medical industry. These graphs include nodes and edges. A node is an item, for example, people, diagnosis, anatomical structure, anatomical abnormalities, etc. An edge label captures the relationship of interests between the nodes, for example, medical history between two people, diagnostic association with an anatomical structure, or diagnostic history between anatomical abnormalities. Additional information can be added to the medical knowledge graph through a combination of input information from a human being or automated and semi-automated methods. The information within the medical knowledge graphs can be understood and deciphered by a human being to ensure quality control.

Natural medical image and/or data refers to any image data captured by a medical imaging capture device or medical imaging acquisition device that is not necessarily formatted according to DICOM standards. These medical imaging capture devices may include conventional X-ray devices, digital radiography X-ray devices, S-ray angiography devices, panoramic X-ray devices, computerized tomography devices, magnetic resonance imaging (MRI) devices, ultrasound imaging devices, and mammography devices. However, these devices are including but not limited to the aforementioned devices.

Additionally, medical images and information sources can include picture archiving and communication systems (PACS). PACS provides convenient access to images from multiple modalities. Electronic images and reports can be transmitted digitally via PACS. This process eliminates manually filing, retrieving, or transporting film jackets and folders used to store and protect X-ray films. The universal format for PACS image storage and transfer is DICOM. Other electronic health record systems include, but are not limited to, Hospital Information System (HIS), Radiology Information Systems (RIS), Laboratory Information Systems (LIS), Clinical Information Systems (CIS), electronic health records (EHR), electronic medical records (EMR).

Annotated medical images may include DICOM images and/or non-DICOM images with forms of annotation or markups, including, but not limited to labeling and measurements. The annotation may include graphical marks, text data, symbol data, tags, areas of interest. In various embodiments, annotated medical images can comprise images generated using AI assisted annotation tools.

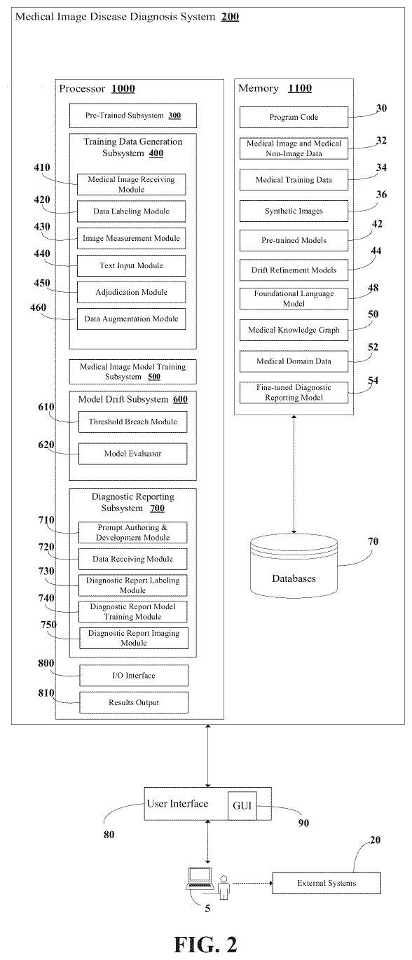

FIG. 2 is a block diagram of a medical image disease diagnostic system 200. System 200 includes at least one processor 1000 (or in some embodiments, a plurality of processors), and memory 1100. Memory 1100 that is in data communication with processor 1000 and typically comprises both non-volatile and volatile memory, including Random Access Memory (RAM), Read only Memory (ROM), and one or more mass storage devices.

As is discussed in greater detail below, processor 1000 includes a pre-trained system 300, a training data generation subsystem 400, a medical image model training subsystem 500, a model drift subsystem 600, and a diagnostic reporting subsystem 700. The training data generation subsystem 400 includes a medical image receiving module 410, data labeling module 420, image measurement module 430, text input module 440, adjudication module 450, and data augmentation module 460. The model drift subsystem 600 includes a threshold breach module 610 and a model evaluator 620. The diagnostic reporting subsystem 700 includes a prompt authorizing and development module 710, data receiving module 720, diagnostic report labeling module 740. Processor 1000 also includes an I/O interface 800 and results output 810.

Memory 1100 includes program code 30, medical image and medical non-image data 32, medical training data 34, synthetic images 36, pre-trained models 42, drift refinement models 44, medical knowledge graph 50, medical domain data 52, and fine-tuned diagnostic reporting model 54.

The medical image disease diagnosis system 200 is implemented, at least in part, by processor 1000 executing program code 30 from memory 1100.

The I/O interface 800 is configured to read or receive medical image and medical non-image data 32, medical training data 34, synthetic images 36, articles data 38, expertise data 40, and/or medical knowledge graph 50 for analysis in one or more of the subsystems in the processor 1000 and training in one or more of the models in memory 1100 (or in both, as discussed in further details below). Analysis results and/or medical images for annotation are presented via the results output 810 and/or GUI 90.

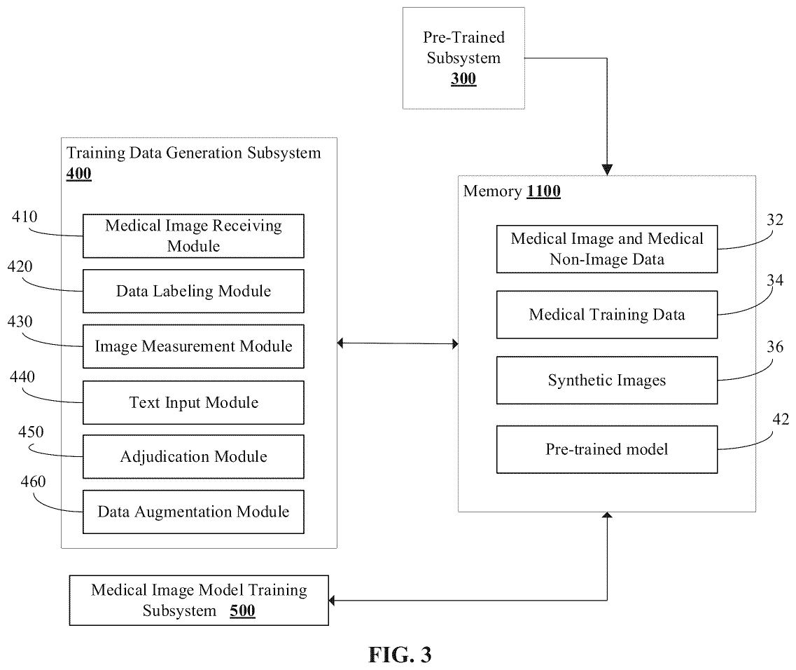

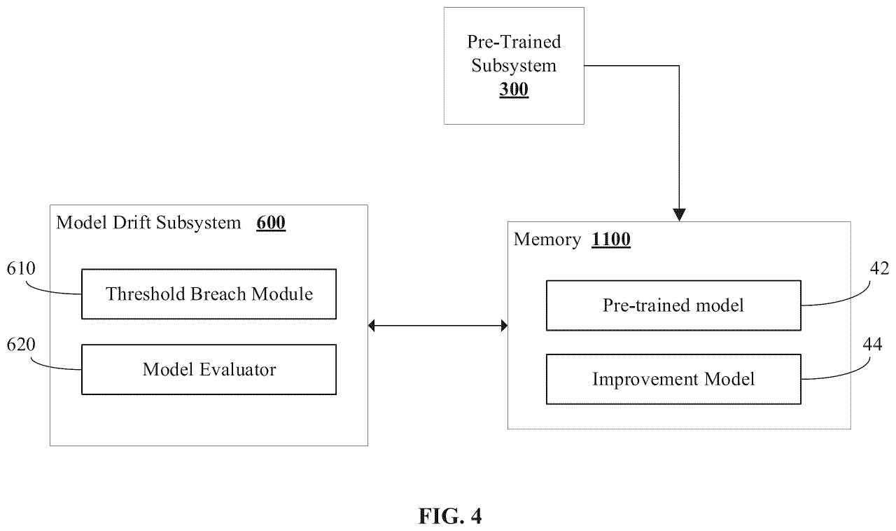

FIG. 3 is a schematic view illustrating a medical training data generation subsystem including the labeling, adjudication, and augmentation of medical images and information according to one embodiment. The medical image receiving module 410 can include software and/or hardware components that facilitate accessing, retrieving, viewing, and reviewing medical image and medical non-image data 32, medical training data 34, and synthetic images 36 in the memory 1100. The pre-trained subsystem 300 includes one or more starting point models that have been trained on a large dataset of medical images and medical non-image information. The pre-trained models are loaded into memory 1100 as the pre-trained model 42. Pre-trained model 42 includes at least one model as the starting point model from the one or more starting point models in the pre-trained subsystem 300. The pre-trained model 42 medical training data that was trained based on DICOM and non-DICOM information.

In some embodiments, annotations tools within the data labeling module 420, image measurement module 430, and text input module 440, respectively, can provide tools for annotating medical images. The medical training data 34 can comprise structured training data that has been annotated by radiologist and/or clinicians according to a format to facilitate effective and consistent machine learning and artificial intelligence model development based on the structured training data. In some implementations, the structured training data can include assembled annotated medical images, medical terms, medical history, identification information (i.e., age, gender). Medical image and medical non-image data 32 is processed by the medical image receiving module 410 and presented to the user 5 via a GUI 90 within the user interface 80. Based on user input via the user interface 80 the data labeling module 420, image measurement module 430, and text input module 440 process the input and store the data as medical training data 34. The data labeling module 420 may receive input from user 5 such as organ identification based on the medical image associated with the patient.

The data labeling module 420 requires clinician input, intelligence, and verification. The labeling performed by the clinician may be information identifying anatomical structures in the region of interest. i.e., heart, lungs, liver, stomach, kidney, the associated abnormalities, i.e., artery defect in the heart, liver lesion, kidney stones, stomach ulcer, and/or the anatomical location within the medical image. Additionally, annotating medical images may include measurements to include the dimension of an anatomical structure, or associated anatomical abnormality, i.e., size of a kidney stone, the measurement of the boundaries surrounding the anatomical structure, and/or distance between identified boundaries that is stored in the medical training data 34 and processed by the image measure module 430. User 5 via the user interface 80 may input textural data in the medical image and medical non-image data 32. The associated text by the clinician may include identifying information associated with the anatomical structure and associated abnormalities, i.e., text input that states the size of the anatomical abnormality, age of the patient, and/or previous medical history. The textural input is stored in the medical training data 34 and processed by the text input module.

Within the workflow of diagnosing a medical disease, clinicians may label, measure, or input data that is incorrect or lacks enough information to effectively diagnosis the medical disease or condition. In the instance of inputting incorrect information, in some embodiments, the clinical workflow may include adjudication by peer or senior clinicians. The annotated medical images and/or medical non-image data is reviewed by peer or senior clinicians to determine the accuracy of the annotation data, i.e., data labeling, measurement, and text input. The medical image and medical-image data 32 and medical training data 34 associated with a patient is presented to the senior clinician via the user interface 80. Discrepancies in the annotation data are corrected by the senior clinician via the adjudication module 450 to correct the associated annotated anatomical data. The adjudication module communicated with memory 1100 and stores the corrected medical training data 34.

In some instances, during the adjudication process, a tie between at least two peer clinicians must be decided upon. When a majority vote must be taken, the adjudication continues with an additional clinician reviewing the annotated medical images and/or medical non-image data performed by the at least two peer clinicians. This adjudication approach allows a majority rule for detection and/or classification tasks. The additional clinician will break the tie to adjust any discrepancies in the annotated data. The additional clinician has expertise and experience higher than the peer clinicians and can adjudicate based on expertise. In this instance, the majority vote process is conducted when there is a need for an odd number of clinicians to break an even number tie.

In some instances, the medical images include noise that makes it difficult to train or retain the pre-trained models. To reduce the noise in medical images, a mean/median approach is used to ensure quality control. For example, measuring the diameter of a kidney stone, the distance between the kidney and neighboring anatomical structure, i.e., spleen, gallbladder, etc., and measuring the diameter of additional kidney stones, applied a quality control to remove noise from the image. This adjudication process ensures that additional data is used to train the pre-trained models. The mean/median approach can use data curation tools to integrate and organize the data. The data curation tools enable data identification, cleansing, and transformation. In this instance, decisions can be made to ensure the right datasets are leveraged to bring value during training and/or retaining of the AI models.

In some instances, during the clinical workflow of diagnosing a medical disease, sufficient medical information or medical images may not be available for the training data generation subsystem 400 to generate the necessary medical training data 34. For example, the medical image may be an image of an anatomical structure that has no associated medical images data in the medical training data 34. In this instance, the medical image may be of a gall bladder that has no associated data in the stored medical image and medical non-image data 32 or the medical training data 34. In the absence of sufficient medical information or medical images, clinicians are not able to effectively diagnosis medical diseases or conditions. In some embodiments, synthetic images 36 may be generated by the data augmentation module 460 from one or more medical images. The clinician and/or senior clinician will identify that accurate annotation is applied to the sufficient medical image and medical non-image data 32 to ensure quality control and confidently diagnosis a medical image. By the execution of computer-executable components associated with the data augmentation module 460 in the processor 1000, and one or more synthetic images 36 is developed and stored in the medical training data 34.

Data augmentation methods performed by the data augmentation module 460 may include, but not limited to, applying transformations that may include dropping, rotating, flipping, scaling, object insertions, magnification, or removing or adding noise. In some instances, multimodal synthetic data is generated from the medical image and medical non-image data 32 by applying modality-specific transformations to base images to simulate the characteristics of desired modality. The transformations can mimic the imaging process, including noise, contrast, and special resolution adjustments. Factors such as acquisition protocols, imaging parameters, and artifacts specific to each modality can be used for modality-specific transformations. The introduction of appropriate noise and artifacts observed in the target modality can be achieved to create synthetic images 36. The methods may be statistical, neural network techniques, such as generative adversarial network (GAN), variational auto-encoder (VAE), or diffusion models, or related random sampling and statistical analysis of the result with the use of Monte Carlo methods.

Validation and evaluation of the synthesized images may be conducted to ensure their realism and fidelity. In some instances, synthetic images can be compared to the real medical image obtained from the corresponding modalities using the three metrics. The three metrics include, but are not limited to, fidelity, privacy, and suitability. Fidelity is how similar synthetic data to the original data. Privacy relates to how easy it is to distinguish information about a real individual from synthetic data. Lastly, suitability relates to how robust a model is on synthetic data and in turn tested on original or real data. The synthetic images 36 are stored in memory 1100. In various embodiments, the data augmentation module 460 can annotate one or more synthetic images 36 with senior clinician input and/or conclusion. In this instance, the senior clinician input and/or conclusion may be considered as the ground truth annotation or labeling for each of the one or more synthetic images 36.

The medical image model training subsystem 500 as illustrated in FIG. 3 trains or creates additional pre-trained models 42 based on one or more of the medical image and medical non-image data 32, medical training data 34, and synthetic images 36. More specifically the medical image model training subsystem 500 can electronically feed the deployed pre-trained models 42 the one or more medical data sets, thereby causing one or more deployed pre-trained models 42 to output results and store memory 1100.

Machine learning models generally suffer from model drift. The one or more pre-trained models 42 was trained with a certain dataset, but over time there may be changes in the data that the pre-trained models 42 is used to analyze. Due to the one or more pre-trained models 42 being trained with old data, it becomes less accurate at performing inference on new data as the data becomes less similar to the medical training data 34. For example, clinicians and/or senior clinicians alter their annotations styles as they adapt to changing medical conditions and industry. As such, new data (e.g., representing the labeling, measuring, or text input) becomes more and more different from the training data.

FIG. 4 illustrates a schematic view of evaluating and improving pre-trained models in the system of one or more embodiments. The model drift subsystem 600 includes a threshold breach module 610 and model evaluator 620. The threshold breach module 610 is trained to recognize discrepancies, i.e., drift, in the performance of the one or more pre-trained models 42. The threshold breach module may be configured to analyze one or more pre-trained models 42 for manual annotations and corrections from the clinician and/or senior clinicians. Based on the number of corrections due to misdiagnosis of medical diseases or conditions by the one or more trained model, the threshold breach model 610 may provide recommendations and/or actions to retrain one or more pre-trained models 42 and/or develop one or more drift refinement models 44. The model evaluator can test one or more drift refinement models 44 using an evaluation dataset to determine whether the one or more drift refinement models 44 performs better than previous version of the one or more pre-trained models 42. If the drift refinement model 44 performs better than older version/versions of the pre-trained model, then the new drift refinement model can be deployed and stored in memory 1100 as a pre-trained model 42.

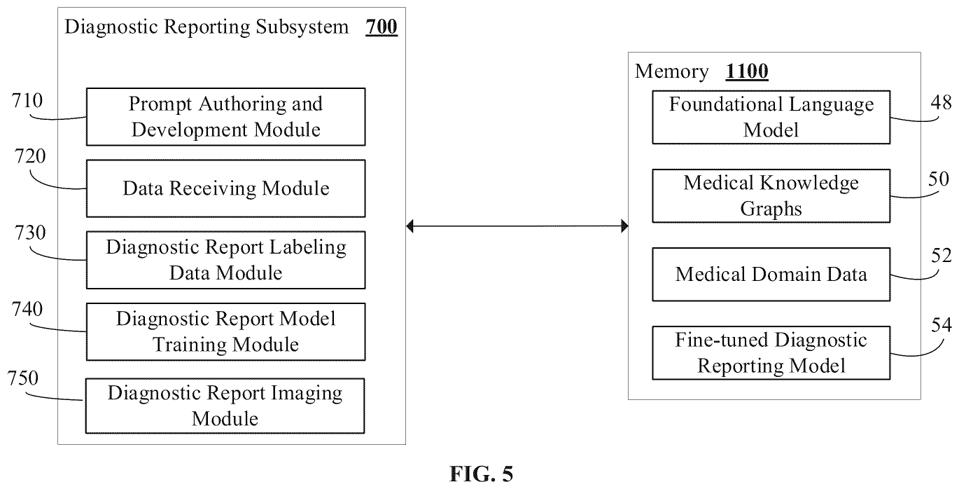

FIG. 5 is a schematic view of developing and retraining diagnostic reporting models in the system of one or more embodiments. According to various embodiments, the diagnostic reporting system 700 includes a prompt authorizing development module 710, data receiving module 720, labeling radiology reporting data module 730, and diagnostic report model training module 740.

Memory 1100 includes a foundational language model 48 that is a large-scale neural network trained on a vast amount of text data. The foundational language model 48 can understand and generate human-like language and include one or more models. In some embodiments, the foundational language model 48 can be one or more pre-trained language models (LMs). The foundational language model 48 is multimodal and can process multiple data types and work in multiple modes, in addition to language.

The foundational language model 48 can be trained on a broad set of data and can be adapted and fine-tuned for medical applications and medical diagnostics, as described in detail below. The foundational language model 48 is one or more pre-trained models that incorporates vast sets of data across a multitude of topics and industries.

Medical knowledge graphs 50 are stored in memory 1100 and include data from multiple sources within the domain of medicine. Medical knowledge graphs 50 includes one or more knowledge bases having data interlinks and nodes within the domain of medicine.

Memory 1100 includes medical domain data 52 that comprises data from multiple data sources. The data sources include medical journals, medical articles, etc. to generate a database of medical domain/expertise terms. The foundational language model 48, as a pre-trained model, incorporates the medical knowledge graphs 50 and medical domain data 52 and generates one or more fine-tuned diagnostic reporting models 54. The fine-tuned diagnostic reporting model 54 can be an AI model of natural language that generates series of words based on texts from the foundational language model 48, medical knowledge graphs 50, and medical domain data 52.

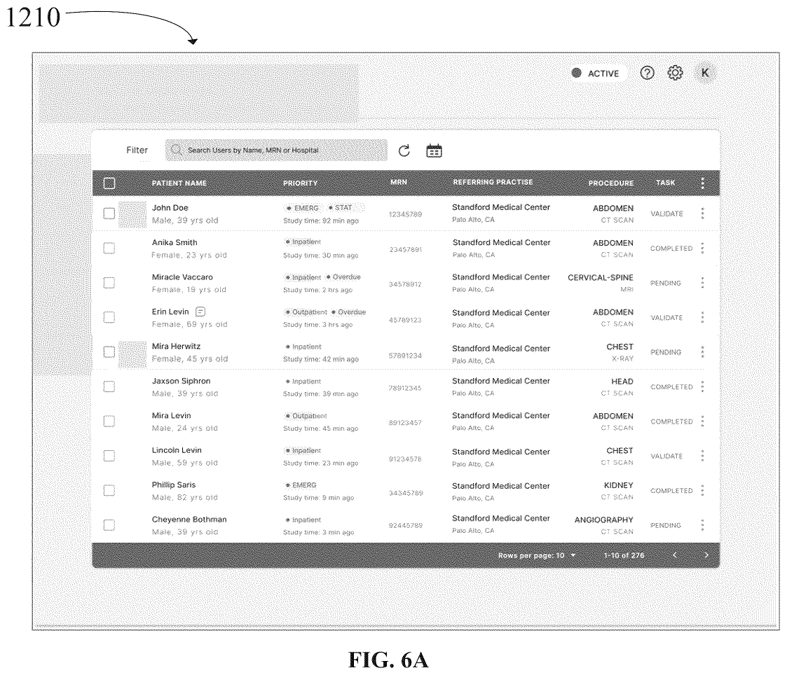

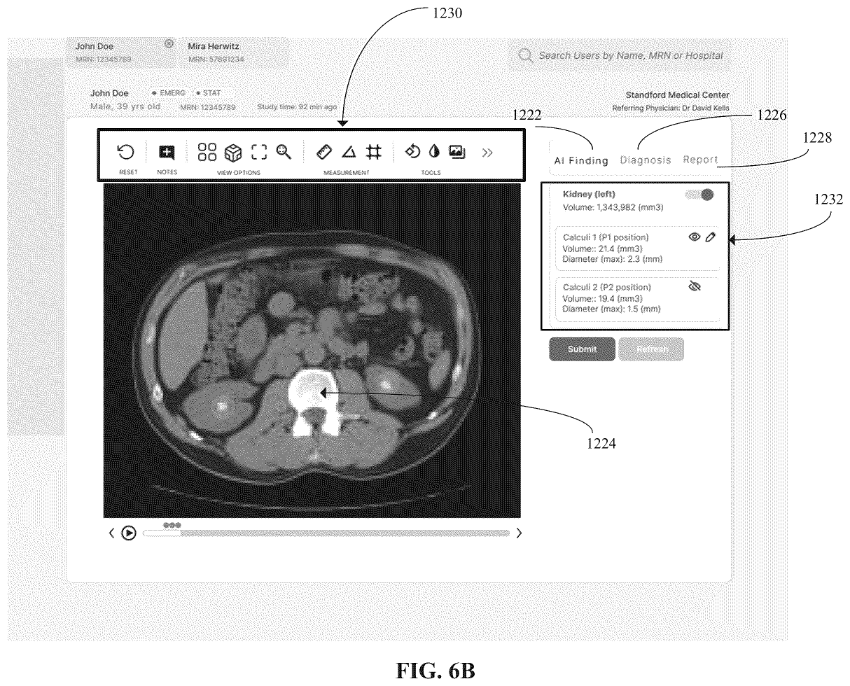

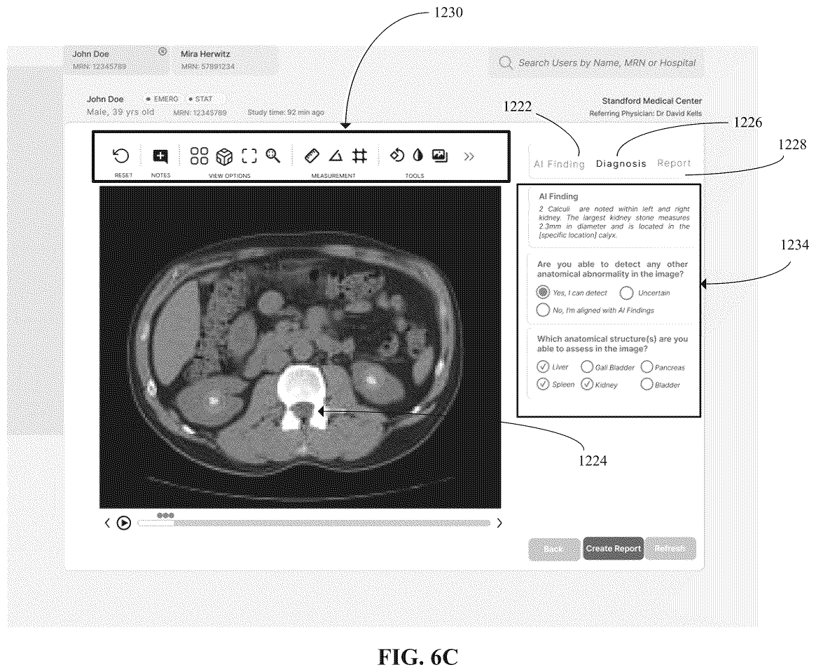

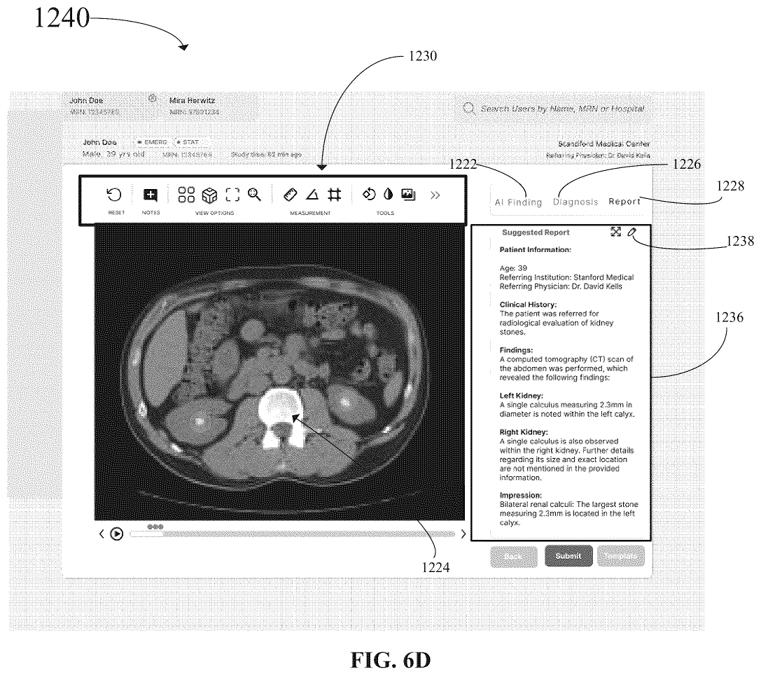

The diagnostic reporting system 700 includes a data receiving module 720 that receives data from the medical image and medical non-image data 32 and medical training data 34, i.e., data from EHR, EMR, HIS, RIS, PACS, etc. The data is incorporated into the diagnostic report model training module 740 to generate one or more fine-tuned diagnostic models 54 to develop a model specific to the patient under evaluation by the clinician. Based on one or more fine-tuned diagnostic models 54, the clinician is displayed a medical imaging visualization on the GUI 90, as shown in FIGS. 6B-6D.

The diagnostic report labeling data module in FIG. 5 incorporates a clinician or senior clinician annotations on medical images and re-trains or creates one or more fine-tuned diagnostic models 54.

Additionally, the clinician or senior clinician is presented on the GUI 90 prompts in a medical image visualization on GUI 90. FIG. 6C illustrates diagnosis prompts panel 1234 with prompts generated by one or more fine-tuned diagnostic reporting models 54, to be described in further detail below. The prompt authoring and development module 710 develops the prompts displayed on the diagnosis prompts panel 1234 based on zero-shot AI generated prompts and/or few-shot prompts that are stored data in nodes and edges of the knowledge graphs.

FIG. 5 includes a fine-tuned diagnostic reporting model 54 is based on tuning techniques applied to the foundational language model 48 incorporating medical knowledge graphs 50, medical domain data 52, and data associated with the patient from the medial image and medical non image data 32, and medical training data 34. The fine-tuned diagnostic reporting model 54 is incorporated into the diagnostic report imaging model 750 to generate a final diagnostic report to the clinician or senior clinician via the GUI 90.

FIG. 6A to 6C present medical imaging visualizations generated associated with integration of one or more diagnostic reporting subsystem modules and one or more pre-trained models with a medical imaging application in accordance with an embodiment of the disclosed invention.

In various embodiments, the medical imaging visualization presented in FIGS. 6A-6D can be generated by the diagnostic report imaging module 750 (or a similar module/application) in association with integration of the features and functionalities provided by one or more the pre-trained foundational language models 48, medical knowledge graphs 50, medical domain data 52, and one or more fine-tuned diagnostic reporting models 54 in accordance with the techniques described herein. Repetitive description of like elements employed in respective embodiments is omitted for sake of brevity.

With reference initially to FIG. 6A, present is an example visualization comprising a display via the GUI 90 visible by clinicians showing a list of available patients to review. The display indicates the patient's name, associated gender, and age to verify the correct patient. Additionally, a priority section is associated with each patient to indicate to the clinician which patient needs evaluation immediately. In one instance, patient John Doe is a 39-year-old male who is currently in the Emergency Room at Standford Medical Center in Palo Alto, CA. The patient underwent an evaluation study approximately 92 minutes prior to the clinician reviewing the visualization.

Additionally, the visualization provides details on the procedure conducted on the patient. In the instance of John Doe, he underwent a CT scan of his abdomen. The clinician can review the status of the patient within the task column. Regarding the current patient, the clinician needs to validate the findings in the CT scan. In other instances, the task can be pending evaluation and findings of the procedure or indication that the task has been completed.

With reference to FIG. 6B, presented is a medical imaging visualization comprising an CT view of a patient CT kidney scan for viewing by the clinician. A kidney calculus 1224 is present in the CT view of the patient. Based on one or more of the foundational language models 48, and medical knowledge graphs 50, the diagnostic reporting subsystem 700 presents via the GUI 70 the AI findings selection option 1222 and AI findings results 1232 to the clinician. For example, based on the AI models, the CT scan produced an image of a left kidney with a volume of 1,343,982 mm³ having a first kidney calculi (stone) in the P1 position, with a volume of 21.4 mm³ and a diameter of 2.3 mm and a second kidney calculi in the P2 position having a volume of 19.4 mm³ and a diameter of 1.5 mm.

Based on the AI findings, the clinician or senior clinician can make any corrections by annotating the image by selecting annotation tools 1230 in response to the diagnostic report labeling data module 730 of the diagnostic reporting subsystem 700. In this instance, the clinician or senior clinician can label the image, add notes, and conduct measurements that will adjust the diagnosis results.

FIG. 6C presents a view of a medical imaging visualization with the diagnosis selection option 1226 selected to present diagnosis prompts panel 1234. In this instance, the clinician or senior clinician can view the AI findings in a summarized view and has prompted options to add associated details in reference to the CT abdomen scan. In response to the selections by the clinician or senior clinician, in this instance the indication of the ability to detect other anatomical abnormality in the presented image and the associated anatomical structures. Based on the AI findings, the diagnostic reporting subsystem 700 presents to the clinician or senior clinician anatomical structures in near range of the kidney, e.g., liver, gall bladder, pancreas, spleen, and bladder. The kidney is presented as an option in case additional anatomical abnormalities are present that were not presented by the AI findings.

In reference to FIG. 6D, the medical imaging visualization presents a report option 1228 for the clinician or senior clinician. Based on the AI findings within the AI findings option 1222 and the diagnosis within the diagnosis option 1226, the diagnostic reporting subsystem 700 presents via the GUI 90 a suggest report display 1236. In this instance, the patient's name, not shown, and associated personal information, e.g., age, referring physician and medical institution is presented based on medical data provided with the CT image. The suggested report display 1236 incorporates the information from the AI findings results 1232 and diagnosis prompts panel 1234 and displays it in natural language for the clinician or senior clinician. For example, the resented suggested report display 1236, a description of the “findings” and an “impression” of the findings is presented. In this regard, the findings indicate that a single calculus measuring 2.3 mm in diameter is noted within the left calyx. The impression indicates that bilateral renal calculi: the largest stone measuring 2.3 mm is in the left calyx. In this embodiment shown, the clinician or senior clinician can further choose to accept and insert the results or reject and edit the results.

In some instances, the clinician or senior clinician can decide that edits to the suggested report is necessary. In this case, the edit command 1238 provides the ability to edit any of the information. In this instance, any information shown in the suggested report display 1236 can be updated, changed, or edited by the clinician or senior clinician. For example, the clinician or senior clinician can update the suggested report to provide more details on the diameter size of the single calculus in the right kidney and remove the description indicating that the size and exact location are not mentioned.

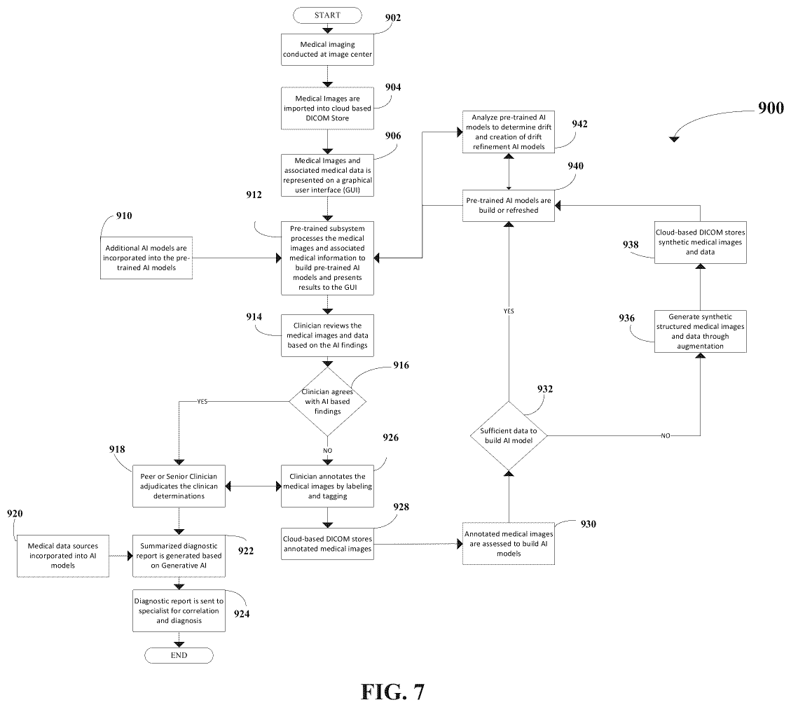

FIG. 7 is a flow diagram 900 of the medical image disease diagnosis system 110. Referring to FIG. 7, at step 902, a medical imaging procedure for a patient is conducted at an image center. After medical images are captured during the medical imaging procedure, at step 904 the medical images are imported into a cloud based DICOM store. Based on the images imported into the cloud based DICOM store, at step 906, medical images and associated medical data is presented to a clinician by a medical imaging viewer on a GUI. The medical image viewer includes advanced tools for image analysis, manipulation, e.g., zoom, pan, rotate, and measurement activities. The pre-trained AI models at step 906 are built outside the clinical workflow. After the completion of the development, the pre-trained models are deployed into the clinical workflow at step 912 into the pre-trained subsystem. The pre-pretrained subsystems processes the pre-trained AI models and presents the results to the graphical user interface at step 912. At step 914, the medical images and data based on the AI findings are presented to the clinician for review.

At step 916 a decision by the clinician must be made to determine agreement with the AI based findings. If the clinician does agree with the findings, processing continues to step 918 for a senior clinician to adjudicate the clinician determinations. At this step, the senior clinician reviews the determination made by the clinician and creates a summarized diagnostic report based on generative AI at step 922. The summarized diagnostic report incorporates medical sources data that has been incorporated into AI models at step 920. During this process, medical data from data receiving sources, such as medical journals, medical literature, is processed by fine-tuned diagnostic reporting models for incorporating into the summarized diagnostic report.

Additionally, during step 920, zero-shot AI generated prompt results and knowledge graph information is incorporated into the AI models. Step 922 incorporates both the senior clinician adjudications and relevant input from step 918 and the AI model results from step 920 to generate the summarized diagnostic report of step 922. At step 924, the diagnostic report is sent to the referring specialist for correlation and diagnosis.

AT step 916, if the clinician does not agree with the AI based findings presented based on step 914, the clinician annotates the medical images by labeling and tagging. During this process, the clinician can label the medical images due to no labeling being presented or correct any labeling provided by the AI models. Additionally, during this process, the clinician can conduct any measurements of the anatomical abnormalities presented by the findings. The annotated medical images are incorporated into the cloud based DICOM stored as indicated by step 928. At step 930 the medial images are incorporated into pre-trained and re-trained AI models at step 930.

At step 932, a decision is made to determine if the data from the annotated medical images is sufficient to build AI models. If the data is sufficient, the annotated medical is used to build or refresh pre-trained AI models at step 940.

The process continues to step 940 to build new or refresh pre-trained AI models. During this process, one or more pre-trained AI models are refreshed based on the new data provided during the annotation process conducted by the clinician. Additionally, in some instances, new pre-trained AI models are created based on the new data provided by the clinician annotations.

The one or more new or refreshed pre-trained models are stored in the pre-trained subsystem of step 912. Additionally, one or more new or refreshed pre-trained models are evaluated and analyzed to determine performance drift at step 942. During this process, the new or refreshed pre-trained models are analyzed to determine if performance of the AI model has decreased over time due to changes, e.g., newly input data based on clinician annotation. Additionally, other changes may cause performance drift, such as changes in the patient population, imaging equipment or clinical protocols.

Based on the analysis results at step 942, drift refinement AI models are created and incorporated into step 940. The drift refinement models are then stored into the pre-trained subsystem at step 912.

If the decision at step 932 is that there is not sufficient data to build AI models, the process of creating a plurality of synthetic structured medical images and data through augmentation is performed at step 936. This process includes applying transformations that may include dropping. rotating, flipping, scaling, object insertions, magnification, or removing or adding noise. Multimodal synthetic data can be generated from the medical image and medical non-image data by applying modality-specific transformations to base images to simulate the characteristics of desired modality. The transformations can mimic the imaging process, including noise, contrast, and special resolution adjustments.

The plurality of synthetic structured medical images and data created at step 936 is incorporated into the cloud based DICOM.

The process continues to step 940 to build new or refresh pre-trained AI models based on the generated synthetic structured medical images and data. During this process, one or more pre-trained AI models are refreshed based on the synthetic medical images and data provided during the augmentation process conducted by the clinician. Additionally, in some instances, new pre-trained AI models are created based on the new data provided by the augmentation process.

The one or more new or refreshed pre-trained models are stored in the pre-trained subsystem of step 912. Additionally, one or more new or refreshed pre-trained models are evaluated and analyzed to determine performance drift at step 942. During this process, the new or refreshed pre-trained models are analyzed to determine if performance of the AI model has decreased over time due to changes, e.g., newly input data based on clinician annotation. Additionally, other changes may cause performance drift, such as changes in the patient population, imaging equipment or clinical protocols.

Based on the analysis results at step 942, drift refinement AI models are created and incorporated into step 940. The drift refinement models are then stored into the pre-trained subsystem at step 912.

The general clinical workflow 900 is a continuous cycle within real-time clinical workflow of medical disease diagnosis. As the overall workflow continues over time, the pre-trained models incorporated as the backbone of the diagnostic process becomes more robust and provides more efficient and accurate diagnosis of medical diseases.

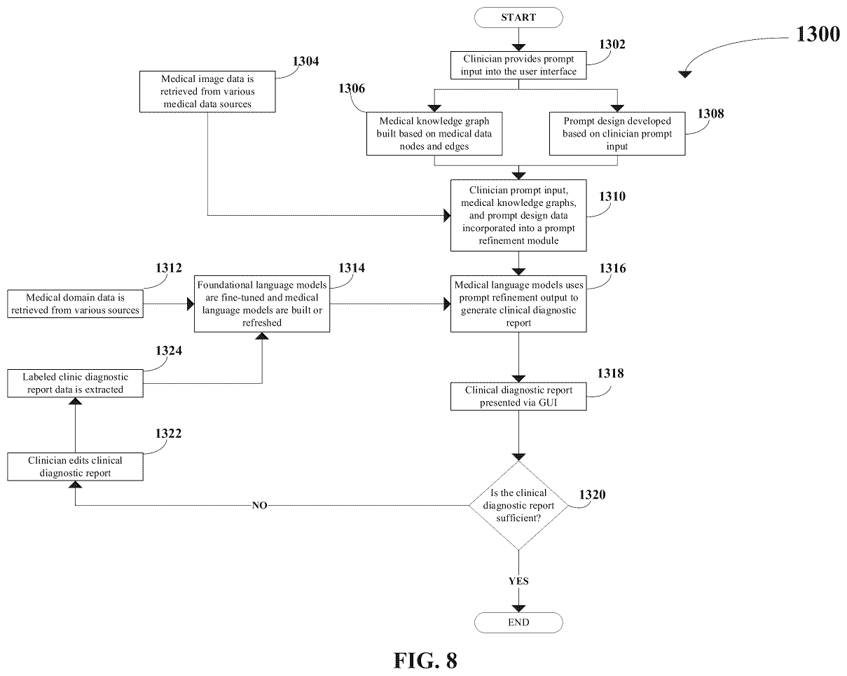

FIG. 8 is a flow diagram 1300 of the diagnostic reporting subsystem 700. Referring to FIG. 8, once a clinician is provided an initial diagnostic report based on the data from the data generation training subsystem 400, at step 1302, a clinician provides input based on prompts presented via the graphical user interface, The prompt input is based on available selections provided via the user interface based on data from the data generation training subsystem in FIG. 2. Once the clinician selects one or more of the available prompt selections, the information is incorporated into medical knowledge graphs. These knowledge graphs have inherent general information based on general and specialized medical data. The knowledge graphs include nodes and edges to show interconnections between the data. The incorporated clinician prompt input tailors the knowledge graphs to a specific patient.

Additionally, at step 1308, the clinician prompt input is further refined by prompt engineering and designing. The prompt engineering and designing process can include processes based on zero-shot AI generated prompts and/or few-shot prompts. In this instance of few-shot prompts, the clinician prompt input includes a small number of examples that guide the model in generating responses for specific tasks. Additionally, in the instance of zero-shot prompts, the clinician prompt input does not include examples.

At step 1310 output from the medical knowledge graph combined with the prompt design data is incorporated into a prompt refinement module. During this process, based on the evaluation, testing, and assessing outcomes, the prompt can be refined, i.e., improved. This process includes updating the instructions for prompt input to be more explicit, incorporating more context or details, changing the phrasing or structure in which the prompt is presented, and modifying prompts to address issues or complications observed during the evaluation process to align with the expected and/or desired outcomes.

At step 1316, one or more medical language models incorporate the output from the prompt refinement output in step 1310 and generate a clinical diagnostic report. In this process, the clinical diagnostic report is generated based on refined prompt input from the clinician and medical knowledge graph data. The clinical diagnostic report is presented to the clinician via a graphical user interface at step 1318. After evaluation of the clinical diagnostic report, the clinician determines if the clinical diagnostic report is sufficient, at step 1320. If the clinical diagnostic report is determined to be sufficient, the process ends with the clinician sending the clinical diagnostic report to the referring physician.

During review of the clinical diagnostic report, the clinician can determine at step 1320 that the report is not sufficient. Based on the clinical diagnostic report not being sufficient, the clinician annotates the clinical diagnostic report step 1322. During this process, the clinician conducts labeling, measuring, and adding textural data to the report, including any medical images within the report. At step 1324, the annotated data, including labeling, measurement, or textural data, is extracted from the clinical diagnostic report.

At step 1314, the extracted data is incorporated into the foundation language models. During this process, the foundational language models are fine-tuned based on the extracted annotated data and medical domain data. Additionally, the foundational language models are further fine-tuned by incorporating medical domain data. The medical domain data can include medical journals, articles, whitepapers, presentations, books, textbooks, and expert speeches or discussions. This specific medical domain data can be applied to foundational language models to adjust the metadata to be specific to the medical field or industry.

In some instances, at step 1314, medical language models are built or refreshed based on the extracted annotated data. Based on the fine tuning of the foundational language models, and/or the building or retraining of the medical language models are refreshed at step 1314 the clinical diagnostic report is fine-tuned at step 1316 and presented to the clinician via the graphical user interface at step 1318. During this process, the clinician reviews the fine-tuned clinical diagnostic report. Based on the review of the report via the graphical user interface, the clinician can determine the fine-tuned clinical diagnostic report is sufficient and sends the report to the referring physician.

It will be understood to persons skilled in the art of the invention that many modifications may be made without departing from the scope of the invention, in particular, it will be apparent that certain features of embodiments of the invention can be employed to form further embodiments.

IT is to be understood that, if any prior art is referred to herein, such reference does not constitute an admission that the prior art forms a part of the common general knowledge in the art of any country.

In the claims which follow and the preceding description of the invention, except where the context requires otherwise due to express language or necessary implication, the world “comprise” or variations such as “comprises” or “comprising” is used in an inclusive sense, i.e., to specify the presence of the stated features but not to preclude the presence or addition of further features in various embodiments of the invention.

What is claimed is:

1. A medical image disease diagnostic system within a clinical medical diagnosis workflow, comprising:
a user interface configured to assess a medical imaging application that provides for one or more activities of viewing, analyzing, annotating, monitoring, or sharing of a plurality of medical images and medical non-image data and medical training data;
a memory, wherein the memory comprises:
the plurality of medical image and medical non-image data,
the plurality of medical training data, and
one or more pre-trained AI models;
a processor, wherein the processor comprises:
a pre-trained subsystem configured to generate the one or more AI pre-trained models based on the plurality of medical image and medical non-image data;
a training data generation subsystem configured to generate the plurality medical training data associated with viewing the plurality of medical images using a medical imaging application; and
a medical image model training subsystem configured to transform the plurality of medical image and medical non-image data into the plurality of medical training data and wherein the medical image model training subsystem is further configured to train or generate the one or more pre-trained AI models based on the plurality of medical training data;
a model drift subsystem configured to determine accuracy of the one or more pre-trained AI models; and
a diagnostic reporting subsystem configured to generate one or more clinical diagnostic reports.

2. The medical image disease diagnostic system of claim 1, wherein the training data generation subsystem further comprises:
a medical image receiving module configured to receive the plurality of medical image and medical non-image data from one or more medical image sources;
a data labeling module configured to generate labeling data based on one or more labeling marks applied to one or more attributes in the plurality of medical images as displayed via the user interface;
an image measurement module configured to generate image measurement data that defines a measurement for one or more attributes in the plurality of medical images; and wherein the image measure data further comprises a dimension of one or more attributes, and wherein the labeling module determines the dimension based on one or more measurement marks; and
a text input module that is configured to apply text to an attribute of interests present in the plurality of medical images displayed via the user interface; wherein the text input module determines one or more text terms that are relevant to the plurality of medical images and generates a picklist of the one or more text terms via the user interface.

3. The medical image disease diagnostic system of claim 2, wherein the medical training data further comprises:
the labeling data;
the image measurement data;
a plurality of location-based data that identifies a location of an attribute of interest relative to the plurality of medical images based on a multi-axis space; and
wherein the medical image model training subsystem is configured to generate or refresh the one or more pre-trained AI models from processing the medical training data.

4. The medical image disease diagnostic system of claim 3, wherein the training data generation subsystem further comprise an adjudication module that is configured to correct one or more of the labeling data, image measurement data, and text data via a user interface.

5. The medical image disease diagnostic system of claim 1, wherein the training data generation subsystem further comprises a data augmentation module that is configured to generate synthetic images comprising multimodal data attributes; and
wherein the multimodal data attributes are determined by the application of modality-specific transformations to the plurality of medical images to generate additional multimodal attributes data.

6. The medical disease diagnostic system of claim 1, wherein the model drift module is further configured to determine the accuracy of the one or more pre-trained AI models by monitoring and tracking the performance of the medical image model training subsystem and generating drift refinement AI models.

7. The medical image disease diagnostic system of claim 3, wherein the system further comprises:
a diagnostic reporting module; wherein the diagnostic reporting module further comprises:
a prompt authoring and development module;
a data receiving module;
a diagnostic report labeling module;
a diagnostic report model training module; and
a diagnostic report imaging module;
and
wherein the diagnostic reporting module is configured to generate textual a clinical diagnostic report regarding medical diagnostic interpretation of the plurality of medical images and medical non-image data; and
train one or more foundational language models based on the textual diagnostic report.

8. The medical image disease diagnostic system of claim 7, wherein the prompt authorizing and development module is configured to generate interactive prompts via the user interface; and
wherein the data receiving module is configured to receive a plurality of medical domain data and a medical knowledge graph and employ artificial intelligence to generate a group of medical terms and medical categories for inclusion in the clinical diagnostic report.

9. The medical image disease diagnostic system of claim 8, wherein the diagnostic report model training module is configured to employ artificial intelligence to fine-tune the one or more foundational language models based on the plurality of medical domain data and the medical knowledge graph; and
wherein the diagnostic report imaging module is configured to generate a fine-tuned diagnostic report.

10. The medical image disease diagnostic system of claim 9, wherein the fine-tuned diagnostic report further comprises interactive prompts generated by employing generative artificial intelligence on the medial domain data and the knowledge graph; and
wherein the interactive prompts are configured to further tune the clinical diagnostic report based on one or more user prompt inputs.

11. A computer-implemented clinical diagnosis method through the utilization of AI-generated information, the method comprising:
training one or more medical image AI model using training data comprising medical images and medical non-image data imported from medical imaging centers to generate medical image results;
presenting the medical image results to a user on a graphical user interface within a user interface, the graphical user interface displaying images based on the medical images and medical non-image data, the medical images generated based on medical image AI model findings;
determining if the medical image AI model findings are accurate;
providing an annotation module that allows the medical image results to be labeled and tagged by user interactions via the user interface, wherein the user interactions result in annotated medical images appearing on the graphical user interface; wherein the annotated medical images are stored in a cloud-based DICOM store;
providing an adjudication module that allows adjudication of the medical images based on the accuracy of the medical image AI model findings and generating a clinical diagnostic report; wherein the clinical diagnostic report includes patient medical information and history.

12. The method as in claim 11, wherein the method further comprises:
assessing the annotated medical images to determine if the annotated data is sufficient to build AI models;
providing an augmentation module that allows synthetic structured medical images to be generated from the medical image results and the annotated medical images; and
storing the synthetic structured medical images in a cloud based DICOM store.

13. The method of claim 12, wherein the synthetic structured medical images comprising multimodal data attributes; and wherein the multimodal data attributes are determined by the application of modality-specific transformations to the medical image results and the annotated medical images to generate additional multimodal attributes data.

14. The method of claim 13, wherein the method further comprises:
comparing the synthetic structured medical images to the annotated medical images to determine the realism of the synthetic structured medical images;
wherein the determination of realism includes ascertaining one of more of fidelity, privacy, and suitability.

15. The method of claim 13, wherein the method further comprises:
training the one or more medical image AI model using one or more of the annotated medical images or the synthetic structured medical images to generate refreshed medical image results;
providing a model drift module that allows tracking of the one or more medical image AI models to determine an accuracy threshold of the one or more medical image AI models;
developing one or more drift refinement AI models based on the accuracy threshold of one or more medical image AI models;
retraining one or more medical AI models based on the one or more drift refinement AI models.

16. The method of claim 11, wherein adjudication comprises:
determining the accuracy of the clinical diagnostic report by reviewing the annotated medical images and based on the accuracy, updating the annotated medical images by an expert user; or
determining the accuracy of the clinical diagnostic report by reviewing an even number or annotated medical images conducted by an even number of users, breaking a tie between the even number of users by updating the annotated medical images in alignment with a single side of the tie; or
determining the accuracy of the clinical diagnostic report by reviewing.

17. A method for producing a clinical diagnostic report from AI generated data, the method comprising:
presenting a plurality of prompts to a user on a graphical user interface via a user interface, the user interface allows one or more of the plurality of prompts to be selected by user interactions, wherein the user interactions produce a prompt input selection result;
developing a medical knowledge graph based on medical domain data; wherein the medical domain data are nodes and edges on the medical knowledge graph;
developing a prompt design based on prompt engineering, the prompt design being produced by interpreting the prompt input selection result; wherein the prompt design produces a refined prompt input;
receiving the knowledge graph and the refined prompt input into a prompt refinement module, the prompt refinement module produces a clinical diagnostic report by medical language models;
presenting the clinical diagnostic report to the user via the graphical user interface; wherein the clinical diagnostic report includes one or more medical images, medical non-image data, patient information, AI generated medical findings, and patient medical history;
evaluating the clinical diagnostic report for accuracy and determining if the clinical diagnostic report has sufficient information to diagnosis an anatomical abnormality.

18. The method of claim 17, including:
determining the clinical diagnostic report is accurate and sending the clinical diagnostic report to an external user; or
annotating the clinical diagnostic report, wherein annotating includes adding annotated data, wherein the annotated data comprises one or more of labeling data in one or more medical images, measuring data of one or more anatomical abnormalities, and textual data, resulting in an annotated clinical diagnostic report.

19. The method of claim 18, further comprising:
extracting the annotated data to generate refined data sets;
training AI language models using refined data sets and medical domain data.

20. The method of claim 19, wherein training AI language models include:
refining at least one or more foundational language models based on the refined data sets to develop refined foundational language models and applying the refined foundational language models to the medical language models to generate refreshed medical language models; and
training the medical language models based on the refined data to generate refreshed medical language models;
developing a clinical diagnostic report based on the refined foundational language models, refreshed medical language models, or medical language models;
presenting the clinical diagnostic report to the user via the graphical interface; and
sending the clinical diagnostic report to an external user.
